(19) United States
(12) Patent Application Publication
(10) Pub. No.: US 2018/0177705 A1
(43) Pub. Date: Jun. 28, 2018

(54) ANESTHETIC MOISTURIZING BODY WAX

(71) Applicant: MONIQUE HAYES, TYRONE, GA (US)

(72) Inventor: MONIQUE HAYES, TYRONE, GA (US)

(21) Appl. No.: 15/390,544

(22) Filed: Dec. 26, 2016

(51) Int. Cl. A61K 8/67; A61Q 9/04; A61K 8/97; A61K 8/92; A61Q 19/08; A61Q 19/00

(52) U.S. Cl. CPC A61K 8/678; A61Q 9/04; A61K 8/97; A61K 2800/805; A61Q 19/08; A61Q 19/00; A61K 8/922

(57) ABSTRACT

The present invention relates to a cosmetic composition added to the body wax wherein, the cosmetic composition is made from naturally ingredients that has properties to treat the at least one users skin. The cosmetic composition herein also contains calming fragrances. The key unique feature of the cosmetic composition is the combination of both an anesthetic and hair gripping/removal properties in a single product. The cosmetic composition includes herbs and essential oils that are anti-inflammatory as well as antimicrobial in nature to provide healthy skin to the at least one user.

### FIELD OF THE INVENTION

The present invention relates to a cosmetic composition for a body wax which is made from a naturally occurring ingredients. The body wax has properties to treat the user's skin and to provide the skin of the user with calming fragrances too. The unique feature in the cosmetic composition for a body wax is the combination of an anesthetic and hair gripping/removal properties in the same composition.

### BACKGROUND OF THE INVENTION

Almost any area of the body can be waxed, including eyebrows, face, pubic area (called bikini waxing), legs, arms, back, abdomen and feet. There are many types of waxing products suitable for removing unwanted hairs. Strip waxing (soft wax) is accomplished by spreading a thin layer of wax over the skin. If soft wax is used; the warmed wax will be applied to the treated area, a cloth strip is pressed onto the wax. Then the strip is quickly removed in the opposite direction of hair growth. The hard-warm wax is placed directly on the skin and left to harden before it is removed with the hairs.

Conventionally, the procedure for hair removal is performed by waxing. This is typically done in a salon by a body wax professional using hot or cold wax applied to the hair the clients wish to have removed. The waxing products are made of petroleum based resins and chemicals which can be prone to contamination or cross contamination. With hot waxing, there are risks of burning skin, damaging the dermal cells, scarring the follicle mouth or surrounding area and causing pigmentation. The wax will adhere to the skin cells, or any surface it is applied to, making pulling it away painful. When waxing, you remove the unwanted hair against the natural direction of growth which causes undue levels of pain and becomes the major contributor to ingrown hair. Hair trapped under the skin can also be painful, unsightly and cause infections. Most of the waxing compositions used in today's market lack a suitable composition for the skin or the body of the at least one user. This could cause discomfort to the at least one user if used extensively. Also most waxing compositions do not contain any agents to lubricate the body of the at least one user; side by side at the time he/she is getting waxed.

Prior art US20130298338A1 discloses an advanced “body sugaring” which is a unique and innovative process for the removal of unwanted facial and body hair for men, women and children. However, the cited art also lacks the novelty feature of an added cosmetic composition for the body wax with an added lubricant element, to sooth the skin and prevent any side effects caused by waxing.

Prior art U.S. Pat. No. 7,527,784B2 discloses a basic hair removal wax made up of water-soluble hair linking agents and further lacks the novelty feature of an added cosmetic composition.

Therefore, there remains a need to overcome above mentioned problems and our invention intends to resolve these problems.

### SUMMARY OF THE INVENTION

To overcome the prior art the present invention provides a cosmetic composition for a body wax. This wax is made from naturally occurring ingredients which have properties to treat the at least one users skin and to provide the skin of the user with calming fragrances. The unique feature in the cosmetic composition for a body wax is the combination of an anesthetic and the hair gripping/removal properties in the same composition.

The product herein is a cosmetic composition added to the body wax wherein, the cosmetic composition is made from naturally occurring ingredients that have a numbing property to moisturize and treat the skin. The cosmetic composition herein contains calming fragrances. The key unique feature of the cosmetic composition is the combination of both an anesthetic and hair gripping/removal properties in a single product. The cosmetic composition includes herbs and essential oils that are anti-inflammatory and antimicrobial in nature; to provide healthy skin to the at least one user.

The cosmetic composition also incorporates vitamin E extracts wherein, vitamin E enables to provide an antioxidant property to the at least one user. Vitamin E herein; works to block free radicals from the body, which play a large part in the aging process. Vitamin E fights off free radicals and hence one is enabled to reduce wrinkles to keep the skin looking youthful.

### BRIEF DESCRIPTION OF THE DRAWINGS

The following drawings present exemplary embodiment; however, they are helpful in illustrating objects, features and advantages of the present invention. The present invention will be more apparent from the following detailed description taken in conjunction with the accompanying drawings in which:

FIG. 1 illustrates the flow chart of the given invention which is also the preferred embodiment according to an embodiment of the invention.

### DETAIL DESCRIPTION OF THE INVENTION

Reference will now be made in detail to the exemplary embodiment(s) of the invention, examples of which are illustrated in the accompanying drawings. Whenever possible, the same reference numerals will be used throughout the drawings to refer to the same or like parts.

References to “one embodiment,” “at least one embodiment,” “an embodiment,” “one example,” “an example,” “for example,” and so on indicate that the embodiment(s) or example(s) may include a particular feature, structure, characteristic, property, element, or limitation but that not every embodiment or example necessarily includes that particular feature, structure, characteristic, property, element, or limitation. Further, repeated use of the phrase “in an embodiment” does not necessarily refer to the same embodiment.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications, patents and patent computer implemented methods mentioned herein are incorporated herein in their entirety.

The product herein is a cosmetic composition added to the body wax wherein, the cosmetic composition is made from naturally occurring ingredients that has a numbing property moisturizing properties and treats the skin. The cosmetic composition herein contains calming fragrances. The key unique feature of the cosmetic composition is the combination of both an anesthetic and hair gripping/removal properties in a single product. The cosmetic composition includes herbs and essential oils that are anti-inflammatory as well as antimicrobial in nature to provide healthy skin to the user.

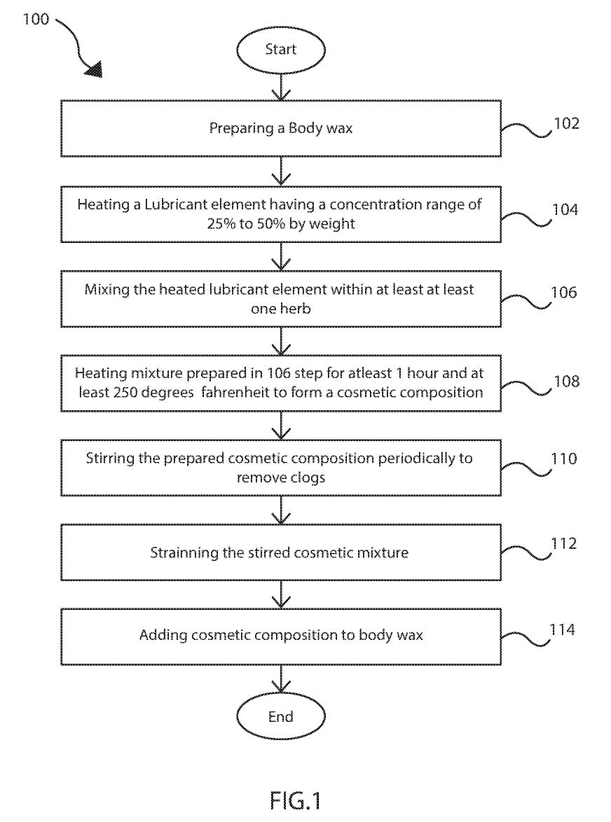

FIG. 1 illustrates a flow chart [100] depicting a method for preparing a cosmetic composition to be added in a body wax.

Step. 102 of FIG. 1 illustrates preparing a body wax. The process of preparing a body wax is accompanied by some simple steps of: combining at least 2 cups of sugar or honey to at least ¼ cup of lemon juice and at least ¼ cup of water to the above prepared mixture. Heating the prepared mixture to 260 degrees Fahrenheit to gain the desired consistency. Once the mixture has reached a syrup consistency it is removed from heat and stored in a container for further process. The wax prepared herein has no discrimination of color, texture and types of skin. It helps in removing any colour or consistency of hair from any type of skin oily, dry or normal. It also helps removing a layer of a dead skin prevailing onto the at least one user body.

Step. 104 of FIG. 1 illustrates heating a lubricant element having a concentration range of 25% to 50% by weight. The cosmetic composition herein is prepared by first heating a lubricant element wherein, the lubricant element is at least one of olive oil, safflower oil, macadamia oil, jojoba oil or the combination thereof. The lack of humidity and cold air causes one's skin to become dry, while in the summer extra exposure to the sun leaves the skin in need of more hydration to keep it moisturized. The best way to keep hydrated, body lotions can be applied however, these creams tend to rub off quickly and leave the skin dry again. Therefore, an alternative to body lotions is this lubricant element present into the cosmetic composition added into the body wax at the time of waxing. The present body wax composition intends to offer the perfect way to keep the skin moisturized and improves one's overall health of the body. In conjunction, the lubricant element present into the cosmetic composition added into the body wax keeps the body hydrated for longer period of time and acts as a substitute for lotions and creams applied to the skin to provide moisture to the body. Furthermore, the lubricating elements used herein are organic in nature, free of chemicals and extracted from roots, fruits, flowers and leaves.

Step. 106 of FIG. 1 illustrates mixing the heated lubricant element with either one of a clove, allspice, kava kava, marjoram or cinnamon having a concentration range of 15 ml to 50 ml by weight. The lubricant element herein is then mixed with the various other elements in perfect proportion wherein, the various other elements mentioned above are classified under herbs and are known for their numbing properties. The various skin benefits of mixing the above-mentioned elements with olive oil are as below:
Treats and removes acne
Cures blemishes and scars
Possesses antioxidant properties
Treats rashes, cuts and wounds
Offers relief from anxiety
Removes stress
Anti-ageing
Helps treat stretch marks or any other marks
Reduces pain associated with waxing
   The above prepared mixture is enabled to be used as a skin cream for treating stiffness and sprains and leave the at least one user with healthy and good looking skin.

Step. 108 of FIG. 1 illustrates heating the mixture prepared in step. 106 for at least one hour and to at least 250 degrees Fahrenheit, in a covered container to form a cosmetic composition. The need for heating the above mixture is explained by the second law of thermodynamics, that is, higher concentration implies lower entropy at some given temperature, and one cannot lower the entropy of an isolated system. It needs to be able to transfer that entropy to another system, and in this case it does so by dissipating heat (and to dissipate heat, it must be given heat). Therefore, in order to provide a proper consistency to the mixture, the mixture is heated at 250 degree Fahrenheit. The temperature provided herein enables to provide proper solubility of the various elements inside the mixture and enables preparing a homogeneous mixture for the same.

Step. 110 of FIG. 1 illustrates stirring the prepared cosmetic composition periodically after heating to remove clogs because the clog formation herein enables to weaken the properties of the cosmetic composition prepared and thereby leads to a reduced effect.

Step. 112 of FIG. 1 illustrates straining the stirred cosmetic composition to separate any solid particles left in the prepared mixture.

Step. 114 of FIG. 1 illustrates adding the cosmetic composition into the body wax.

In another embodiment the cosmetic composition is also added with extracts of vitamin E wherein, vitamin E is enabled to provide an antioxidant property to the at least one user. Vitamin E herein works to block free radicals from the body, which helps as an anti-aging agent. Vitamin E fights off free radicals and hence one is enabled to reduce wrinkles to keep the skin youthful-looking. It has basic antioxidant properties that everyone needs. The various benefits of having vitamin E as one of the elements inside the cosmetic composition are as below:
Acts as a moisturizer
Reverses premature aging of the skin
Treats sun burns
Lightens dark spots
Acts as a cleansing element
Treats stretch marks
Soothes pain from cold sores

While the invention has been described in detail with specific reference to preferred embodiments thereof, it is understood that variations and modifications thereof may be made without departing from the true spirit and scope of the invention.

What is claimed is:

1. A method for preparing a cosmetic composition to be added in a body wax comprises the steps of:
a. heating a lubricant element having a concentration range of 25% to 50% by weight;
b. mixing the heated lubricant element with either one of a clove, allspice kava kava, marjoram, or cinnamon having a concentration range of 15 ml to 50 ml by weight;
c. heating a mixture prepared in step b. for at least 1 hour and to at least 250 degrees Fahrenheit in a covered container to form a cosmetic composition;
d. stirring the prepared cosmetic composition periodically after heating to remove clogs;
e. straining the stirred cosmetic composition in order to separate any of solid particles left in the prepared mixture; and
f. adding the cosmetic composition into the body wax.

2. The cosmetic compositions for a body wax in claim 1, wherein the cosmetic compositions for a body wax further comprises a hair gripping and a hair removal property.

3. The cosmetic compositions for a body wax in claim 1, wherein cosmetic compositions for a body wax is also enabled to be added with extracts of vitamin E to provide an antioxidant property.

4. The cosmetic compositions for a body wax in claim 1, wherein the clove, allspice, kava kava, marjoram or cinnamon mixture with the lubricant element is a substantially miscible and forms a homogeneous mixture.

5. The cosmetic compositions for a body wax in claim 1, wherein the cosmetic compositions is anti-inflammatory as well as antimicrobial in nature to provide a healthy skin to at least one user.

6. A method for preparing a cosmetic composition to be added in a body wax comprises the steps of:
a. heating a lubricant element having a concentration range of 25% to 50% by weight;
b. mixing the heated lubricant element with either one of a clove, allspice, kava kava, marjoram or cinnamon having a concentration range of 15 ml to 50 ml by weight;
c. heating a mixture prepared in step b. for at least 1 hour and to at least 250 degrees Fahrenheit in a covered container to form a cosmetic composition;
d. adding extracts of vitamin E to the prepared cosmetic composition;
e. stirring the prepared cosmetic composition periodically after heating to remove clogs;
f. straining the stirred cosmetic composition in order to separate any of solid particles left in the prepared mixture; and
g. adding the cosmetic composition into the body wax.

7. The cosmetic compositions for a body wax in claim 6, wherein the cosmetic compositions for a body wax further comprises a hair gripping and a hair removal property.

8. The cosmetic compositions for a body wax in claim 6, wherein the extracts of vitamin E to are enabled to provide an antioxidant property.

9. The cosmetic compositions for a body wax in claim 6, wherein the clove, the allspice, the kava kava, the marjoram, the cinnamon mixture with the lubricant element is substantially miscible and forms a homogeneous mixture.

10. The cosmetic compositions for a body wax in claim 6, wherein the cosmetic compositions are anti-inflammatory as well as antimicrobial in nature to provide healthy skin to at least one user.
